(19) United States
(12) Patent Application Publication
(10) Pub. No.: US 2004/0152191 A1
(43) Pub. Date: Aug. 5, 2004

(54) Process for the maturation of dendritic cells and for the activation of macrophages with RU 41740

(75) Inventors: Dominique Rigal, Saint Didier Mont d'Or (FR); Daniel Masseau, Us (FR)

Correspondence Address: MERCHANT & GOULD PC, P.O. BOX 2903, MINNEAPOLIS, MN, 55402-0903, US

(73) Assignee: L C O Sante, Osny (FR)

(21) Appl. No.: 10/616,868

(22) Filed: Jul. 10, 2003

Related U.S. Application Data
(63) Continuation of application No. PCT/FR02/00088, filed on Jan. 10, 2002.

(30) Foreign Application Priority Data
Jan. 10, 2001 (FR) 01/00275

(51) Int. Cl. C12N 5/08

(52) U.S. Cl. 435/372

(57) ABSTRACT

The invention concerns methods for maturation of dendritic cells by contacting them with RU 41740 or a compound analogous to RU 41740. The maturation of dendritic cells contacted with RU 41740, or analogues thereof, can be characterized by their functional properties and their phenotypic properties.

The present invention is situated in the field of cell therapy. It relates to a process capable of generating mature dendritic cells and/or activated mammalian macrophages starting from monocytes, monocyte precursors or hematopoietic stem cells.

The dendritic cells play a critical role in the emergence of the anti-tumor, anti-infectious and auto-immune immune response. In fact, the dendritic cells are the only cells capable of inducing a primary response from the T lymphocytes. They thus have a key role in the initiation of the immune response. Such a function is related to the many morphological properties and surface molecules of the dendritic cells. In fact, owing to extensive increases in size of their cytoplasmic membrane, the dendritic cells have a particularly large contact surface with their environment. Furthermore, they possess at their surface very many histocompatibility antigens of class I and class II which makes antigen presentation possible.

After being taken up by the dendritic cell, the antigen undergoes “processing” before being presented to the T lymphocyte. The re-organization which requires an active cell metabolism comprises four steps: the capture of the antigen, its enzymatic degradation to small peptide fragments in an intracellular compartment, the association of these fragments with the class II molecules of the MHC and the migration of the peptides-class II molecules complexes to the surface of the antigen presenting cell (APC) for presentation to the T cell receptor (TcR) of the helper T lymphocyte (Th). The first step (capture), which is temperature-independent, is performed through the intermediary of non-specific receptors or as a result of other mechanisms still poorly understood. The larger the molecules, the more easily they are captured. The second step, which is temperature-dependent (it is blocked at 4 degrees), is the_ingivitis_tion of the antigen in the phagosomes which then fuse with the lysosomes (intracytoplasmic organelles rich in proteases), thus giving rise to the endosomes at acid pH. The proteolysis of the antigen into peptide fragments occurs in these vesicles as a result of the action in particular of cathepsin B, then D. This step can be blocked by ammonium ions which inhibit the phagosome-lysosome linkage, or by chloroquine which raises the pH of the lysosomes. The third step is a complex process still poorly understood in detail, which results in the association between the class II molecule of the MHC and some specific fragments of the degraded antigen usually constituted of nine to twenty five amino acids. The fourth step involves the migration of the class II molecule-peptide complex (called C3 form) to the surface of the APC. The complex thus formed can then interact with the appropriate TcR at the surface of the Th lymphocyte provided that the molecules of the major histocompatibility complex (MHC) of the lymphocyte belong to the same haplotype as those of the presenting cell (allogenic restriction).

The dendritic cells are moreover very rich in co-stimulatory molecules of the immune response, such as the molecules CD80, CD86, CD40 which activate the molecules CD28, CTLA-4 and CD40L of the T lymphocytes, respectively, by initiating the immune response. They also possess very many adhesion molecules, like the molecule CD54 or the molecule CD11a/CD18, and this facilitates the co-operation between the dendritic cells and the T cells. Another special characteristic of the dendritic cells is to deploy different functions depending on their stage of differentiation. Thus, the capture of the antigen and its transformation are the two principal functions of the immature dendritic cell, whereas its capacities to present the antigen in order to stimulate the T cells increase as the dendritic cells migrate into the tissues and the lymphatic ganglia. This change of functionality corresponds to a maturation of the dendritic cell. Thus, the passage of the immature dendritic cell to the mature dendritic cell represents a fundamental step in the initiation of the immune response. This maturation can be easily followed owing to the change of the surface markers during this process. The surface markers characteristic of the different stages of maturation of the dendritic cells are summarized in the Table below.

TABLE 1

| Cell type | Surface markers |
|---|---|
| Monocytes | CD14++, DR+, CD86+, CD16+/−, CD54+, CD40+ |
| Immature | CD14−, CD16−, CD80+/−, CD83−, CD86+, CD1a+, |
| dendritic cell | CD54+, DQ+, DR++ |
| Mature | CD14−, CD83++, CD86++, CD80++, DR+++, |
| dendritic cell | DQ++, CD40++, CD54++, CD1a− |

The isolation of the dendritic cells from peripheral blood is very difficult because less than 1% of the white blood cells belong to this category. In the same way, the extraction from the tissues is impossible in man and very complicated in animals. That is why an important advance was made when it became possible to generate dendritic cells from hematopoietic precursors and monocytes in the presence of different cytokines. Immature dendritic cells can be produced from monocytes in the presence of GM-CSF and IL-4 and the immature dendritic cells obtained will mature after contact with the TNF-α or with other agents such as the CD40 ligand, the LPS or media conditioned with macrophages. These last agents are complex or toxic substances. Similarly, the activation of the macrophages in vivo is complicated and difficult to control. That is why the activation ex vivo represents an appropriate means for experimental studies and therapeutic applications.

The activated macrophages are cells that are encountered in the tissues after a process of inflammatory activation by specific or non-specific inducers. These cells are involved in the removal of toxic or pathogenic agents or cancerous or degenerate cells.

RU 41740, sold under the trade name Biostim by the Cassenne Laboratories (France), is a medicine composed of glycoprotein extracts obtained from a strain of Klebsiella pneumoniae K₂O₁ (strain O1K2 NCTC 5055). It is obtained after lysis of the bacterial cell walls, organic extraction, centrifugation and ultrafiltration.

It is composed of: −80% of glycoproteins

c) amino acids, lipids and nucleic acids.

The glycoprotein part is divided into 3 fractions: P1, F1, F2.

P1, of capsular origin, represents about 50% of RU 41740 and has a mean molecular weight of 95 kD.

F1 is of membrane origin and represents about 20% of RU 41740 and has a mean molecular weight of 350 kD.

F2 seems to be a part of P1.

RU 41740 does not cause a pyrogenic effect as is demonstrated by the negative Limulus test performed with this compound.

The present invention describes a novel procedure for the maturation of the dendritic cells by using as inducing agent of this maturation, RU 41740 or an analogue of this latter as defined below.

Of the agents making possible the maturation of the dendritic cells, the TNF-α is the one whose activity has been best characterized. Unfortunately, this lymphokine is very toxic and can trigger extremely violent responses in vivo, and this presents a major obstacle to its use in cell therapy. The LPS is another compound capable of inducing the maturation of the dendritic cells. It also exhibits great toxicity and induces a powerful pyrogenic effect, which is demonstrated by a positive Limulus test carried out with the LPS. It produces in addition variable results depending on the batch used. Finally, the LPS possesses the disadvantage of being degraded very rapidly. As regards the ligand of CD40 (CD40L), it directs the differentiation of the dendritic cells in a variable manner depending on the concentration and the time of incubation, and this makes it difficult to use in cell therapy.

As illustrated in the experimental examples below, RU 41740 makes it possible to induce the maturation of the dendritic cells with an efficacy similar to or better than the reference agents cited above. In addition it possesses several significant advantages compared with these agents. In particular, RU 41740, sold under the trade name Biostim, has been used since 1982 as a medicine to stimulate the immune system during chronic infections (chronic bronchitis, otitis, rhinitis . . . ), in a curative or preventive capacity. Its perfect tolerance by the organism has been demonstrated. Furthermore, RU 41740 is an extremely stable compound and the inventors have shown that it induces the maturation of the dendritic cells in a very reproducible and dose-dependent manner.

In a perspective of cell therapy requiring mature dendritic cells, RU 41740 is thus particularly valuable, owing to its lack of toxicity and simplicity of use in association with its stability and the reproducibility of the results obtained.

The use of any analogue of RU 41740 exhibiting similar properties to the latter in processes such as those described below, is obviously included in the framework of the present invention. In what follows, a compound comprising 60 to 90% of glycoproteins, and capable of inducing a significant increase of the expression of the molecules CD40, CD83, CD86 and HLA-DR and a very considerable diminution of the expression of the molecules CD14 and CD1a by the said dendritic cells when placed in contact with immature dendritic cells at concentrations less than or equal to 1 mg/ml will be called an “RU 41740 analogue”. Examples of RU 41740 analogues are LCOS 1013 and LCOS 1014, the processes for the production of which are described in Example 10. In the remainder of the text, unless otherwise indicated, the term “RU 41740” will designate both RU 41740 itself, constituting the active principle of Biostim, and an analogue of the latter.

A “RU 41740 analogue” is defined here as a compound consisting of glycoprotein extracts obtained from a Klebsiella strain (for example, the strain O₁K₂NC TC 5055 of Klebsiella pneumoniae) as a result of at least the following steps:

culture of the strain

lysis of the bacterial cell walls

organic extraction

centrifugation

ultrafiltration

drying

Example 10 below presents two processes for the production of RU 41740 analogues, designated under the references LCOS 1013 and LCOS 1014. The chemical structure of a RU 41740 analogue is composed mainly of

carbohydrates=70%±12

protein=20%±6

Lipids, nucleosides and amino acids are present in trace amounts.

RU 41470 or an analogue of the latter possesses undetectable levels of bacterial endotoxins (below 10 pg/ml).

RU 41470 is active in all of the processes using monocytes, precursors of the monocytes or hematopoietic stem cells in man and animals. Furthermore, RU 41740 makes it possible to obtain at the same time activated macrophages from the same starting cells.

The maturation of the dendritic cells as a result of placing them in the presence of RU 41740 can be demonstrated by their phenotypic properties or by their functional properties.

Thus, the invention relates to a process for obtaining mature dendritic cells (Monocyte DendriticCells, or MODC) or activated macrophages, starting from monocytes, monocyte precursors or hematopoietic stem cells, characterized in that the said monocytes, monocyte precursors or stem cells are placed in contact with RU 41740 or analogue of the latter, this compound being selected such that the placing in contact of immature dendritic cells with the said compound makes possible the functional maturation of the dendritic cells, as demonstrated by their capacity to:

c) trigger in vitro a primary response against an infectious or tumor antigen placed in contact with the dendritic cells beforehand and/or during their culture with the T lymphocytes;

c) induce the proliferation of T lymphocytes in a mixed autologous or allogenic culture

Examples 6, 7, 9, 11 and 12 are illustrations of this process.

The invention also relates to a process for obtaining mature dendritic cells or activated macrophages starting from monocytes, monocyte precursors or hematopoietic stem cells, characterized in that the said monocytes, monocyte precursors or stem cells are placed in contact with RU 41740 or an analogue of this latter, this compound being selected such that the placing in contact of immature dendritic cells with the said compound makes possible the phenotypic maturation of the dendritic cells, demonstrated by a significant increase in the expression of the molecules CD40, CD83, CD86 and HLA-DR and a considerable diminution of the expression of the molecules CD14 and CD1a by the said dendritic cells.

Examples 1 to 3 are illustrations of this process.

Moreover, the natural physico-chemical properties of RU 41740 make it possible to adsorb molecules to them, in particular antigenic molecules. Hence this makes it possible to carry out in a simple manner a non-covalent coupling between RU 41740 and antigenic molecules. It will then be possible to obtain mature dendritic cells specific for the antigens adsorbed to RU 41740 or an analogue of the latter by incubating immature dendritic cells with a coupling product formed between RU 41740 or its analogue and the antigenic molecules selected. The coupling between RU 41740 or an analogue of the latter and the antigenic molecules can be carried out by any procedure known to the specialist skilled in the art. In a preferred manner, the antigens will be adsorbed to the surface of RU 41740 or its analogue, but other means of coupling (covalent linkage, affinity, etc. . . . ) can also be envisaged. The simplest coupling procedure, by adsorption to the surface of RU 41740 also possesses the advantage of making possible a coupling with essentially non-protein antigenic molecules.

The product of coupling between RU 41740 or an analogue of the latter and antigenic molecules in order to induce the maturation of dendritic cells or the activation of macrophages forms part of the present invention. In a preferred embodiment of the coupling products of the invention, the coupling is assured by a non-covalent linkage. A particular coupling product is that of RU 41740 or an analogue of the latter with an essentially non-protein antigenic molecule.

The invention also relates to a process for obtaining mature dendritic cells presenting selected antigens, starting from monocytes, monocyte precursors or hematopoietic stem cells, characterized in that the said monocytes, monocyte precursors or stem cells are placed in contact with RU 41740 or an analogue of the latter, coupled to molecules comprising the said antigens.

In a preferred embodiment of the invention, the processes described above make it possible to obtain mature dendritic cells or activated macrophages by placing monocytes, monocyte precursors or hematopoietic stem cells in contact with RU 41740, coupled or not to antigenic molecules.

In a preferred embodiment of the processes of the invention, RU 41740 is added to the culture medium of the cells at a final concentration included between 1 ng/ml and 1 mg/ml, and preferentially between 100 ng/ml and 10 μg/ml.

In another preferred embodiment of the procedures of the invention, the monocytes, monocyte precursors or stem cells are placed in contact with an analogue of RU 41740 obtained from the strain O₁K₂ NCTC 5055 of Klebsiella pneumoniae. Such an analogue is for example LCOS 1013 or LCOS 1014, which is added to the culture medium of the cells at a final concentration preferably included between 1 ng/ml and 1 mg/ml and, in an even more preferred manner, between 100 ng/ml and 50 μg/ml. In the processes of the invention, the time of incubation of the monocytes, monocyte precursors or hematopoietic stem cells in the presence of RU 41740 or an analogue of the latter is preferentially from 1 to 15 days.

Cell therapy is a recent approach which consists of administering to a patient cells modified ex vivo so as to confer on them properties likely to be beneficial for the patient. The natural properties of the dendritic cells make them an excellent candidate for approaches to cell therapy in several fields of pathology, owing to their capacity to induce a primary immune response from the T lymphocytes. In particular, anti-tumor immunotherapy by administration of dendritic cells presenting one or more tumor antigens is a particularly promising cell therapy approach. The principle of this approach is to present to the immune system tumor antigens in a particularly efficacious manner in order to stimulate a response against the cells presenting the antigens. This approach is illustrated in Example 6 below.

Example 7 presented below demonstrates that the administration of dendritic cells presenting antigens of micro-organisms makes it possible to obtain a primary immunization against these micro-organisms and can hence be used to combat infection.

Another cell therapy approach using dendritic cells consists of directing the dendritic cells to express a tolerance reaction of the host towards particular antigens. This can be useful for example in order to induce a tolerance towards alloantigens at the time of an allogenic graft, to autoantigens at the time of an autoimmune disease, or to allergens at the time of an allergic disease.

Indeed, the dendritic cells, under certain culture conditions, can cause an anergic reaction, i.e. the functional inactivation of the T lymphocytes instead of their activation. The culture of the dendritic cells in the presence of immunosuppressants like cyclosporin or histamine leads to a modification of the membrane antigens which will induce a tolerance response and not a cytotoxic response. This observation could have important implications in the context of organ grafts on the one hand and in the context of the treatments of the auto-immune diseases, on the other, like rheumatoid polyarthritis, myasthenia, insulino-dependent diabetes, multiple sclerosis, eczema, psoriasis, etc. and the allergic diseases. Example 8 illustrates this approach by showing the influence of cyclosporin A in the presence of RU 41740 on the maturation of the dendritic cells.

Whatever the type of pathology concerned, the cell therapy can be carried out by administering to the human or animal patient autologous, homologous or xenologous cells after their modification ex vivo.

The present invention thus relates to a process such as those described above in which the dendritic cells are treated ex vivo and administered after maturation to a human or animal patient in an autologous, homologous or xenologous manner for the prophylaxis, attenuation or treatment of cancerous, infectious, allergic or auto-immune diseases.

The use of RU 41740 or an analogue of the latter for the preparation of a composition comprising mature dendritic cells and/or activated macrophages and/or Langerhans cells of the skin, are also included in the scope of the present invention.

The inventors have shown that RU 41740 makes it possible to induce in vitro the maturation of Langerhans cells (Example 4). This property could thus be advantageously used to promote an immune response at the level of the skin or mucus membranes by topical administration of a composition containing RU 41740. Examples of indications for such a composition are in particular the _ingivitis and the parodontites. The use of RU 41740 or an analogue of the latter for the preparation of a pharmaceutical composition for a topical or systemic administration hence also form part of this invention.

In another aspect of the invention, RU 41740 or an analogue of the latter is coupled to one or more antigens of interest, then administered directly in vivo in order to induce the production by the organism of mature dendritic cells or activated macrophages presenting the said antigens. In this realization of the invention, RU 41740 or its analogue serves in fact as vector for the antigens of interest, and makes possible the presentation of these antigens to the immune system such that it induces the production of mature dendritic cells or activated macrophages presenting the said antigens.

Hence the invention relates to a process such as those described above in which the mature dendritic cells or the activated macrophages are produced directly in vivo.

The use of a coupling product formed between RU 41740 or an analogue of the latter and one or more antigens for the preparation of a composition able to induce the production of mature dendritic cells or activated macrophages presenting the said antigens are also included in the scope of this invention.

The mature dendritic cells obtained by processes such as those described above can be used in the treatment of various types of pathologies, in particular in anti-tumor immunotherapy, in the effort to combat infection or in order to increase the tolerance of the organism towards certain specific antigens. In a preferred embodiment of the invention, the dendritic cells obtained by a process such as described above are used in the production of a composition able to promote an anti-tumor immune response.

Similarly, the use of dendritic cells obtained by a process of the present invention in the production of a composition able to promote an immune response against an infection by a micro-organism is an integral part of the invention.

In another preferred realization of the invention, the dendritic cells obtained by a process such as described above are incubated in the presence of an immunosuppressant and used in the production of a composition able to modify the immune response in the sense of a tolerance.

The mature dendritic cells produced by the processes of the invention can also be used to identify minor histocompatibility antigens. This technique consists of recovering the monocytes by making them differentiate into mature dendritic cells, then by using them as stimulant cells in a mixed lymphocyte culture reaction between individuals of the same compatible HLA family. This system makes it possible to detect disparities concerning the minor histocompatibility antigens and to be able to investigate in depth the compatibility or the incompatibilities between persons of the same family, and this presents definite advantages in the field of intra-familial or even extra-familial tissue and organ grafts. The use of dendritic cells obtained by a process of the invention for the detection and/or the characterization of the histocompatibility antigens also forms part of the present invention.

The Examples and Figures presented below as non-limiting will make it possible to demonstrate certain advantages and characteristics of the present invention.

### LEGENDS TO THE FIGURES

FIG. 1 shows the change of the cell markers during the differentiation of the monocytes into dendritic cells on D0 (dotted curves=monocytic markers before treatment), D6 (curves in fine lines=immature dendritic cells) and D8 (curves in thick lines=mature dendritic cells) after addition of RU 41740 at 25 μg/ml from D6 onwards.

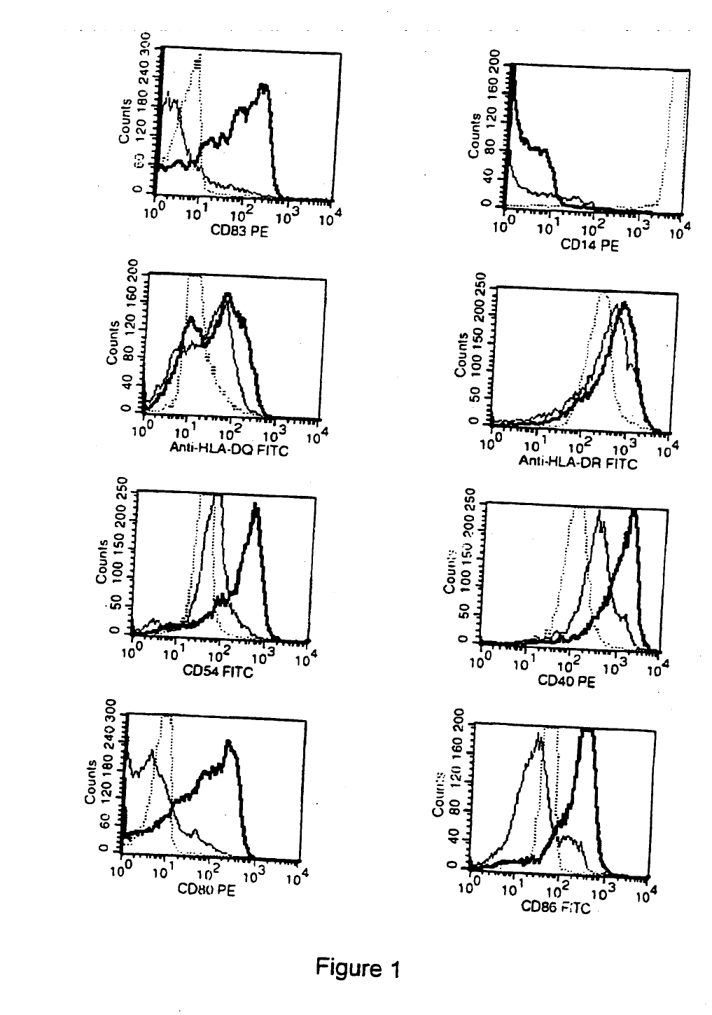

FIG. 2 illustrates the generation of cytotoxic T lines specific for the thyrocalcitonin peptide. The effector/target ratio used is indicated along the abscisse, whereas the percentage lysis of the target cells is indicated along the ordinate.

FIG. 3 demonstrates the presentation of the tetanus toxin by the dendritic cells derived from human monocytes cultivated in the presence of GM-CSF, IL-4 and RU 41740. The incorporation of tritiated thymidine measured in counts per minute (cpm) is indicated along the ordinate.

FIG. 4 illustrates the influence of cyclosporin A (CsA) on the maturation of the dendritic cells derived from monocytes cultivated in the presence of GM-CSF, IL-4 and RU 41740. The curves obtained in flow cytometry show the expression of CD83 in the absence of CsA (4A) and in the presence of CsA at 5 μg/ml (4B). The FIGS. 4C and 4D show the same curves for another marker of the dendritic cells, the DC Lamp antigen. The dotted curves were obtained with an anti-KLH primary antibody, not relevant for the dendritic cells.

FIG. 5 shows the orientation of the immune response towards a type Th2 immune response by the dendritic cells derived from monocytes and treated with cyclosporin A (CsA). The ordinate represents respectively the secretion of IL-12 (FIG. 5A) and the ratio of the secretion of cytokines IFN-γ/IL-10 (Th1/Th2) (FIG. 5B) by dendritic cells derived from monocytes cultivated in the presence of GM-CSF, IL-4 and RU 41740, and treated with cyclosporin A at 5 μg/ml (black columns) or untreated with cyclosporin A (white columns). Three experiments are shown for each condition. The results are presented in percentages with respect to the monocytic cells not treated with cyclosporin, the measurements of which were adjusted to 100.

FIG. 6 shows the results of mixed allogenic lymphocytic reactions carried out with dendritic cells obtained from purified monocytes of three different donors. Two types of dendritic cells were compared, the maturation of which was induced either by TNF-α (200 U/ml) or by LCOS 1013 (25 μg/ml) (curve marked “LCOS”) for 48 hours. The horizontal axis corresponds to the number of irradiated dendritic cells deposited in each well, for a constant quantity of 10⁵ T lymphocytes per well. The proliferation of the T lymphocytes, stimulated by the dendritic cells, is determined after 4 days of culture by the incorporation of tritiated thymidine (vertical axis).

FIG. 7 shows the results of mixed autologous lymphocytic reactions, carried out with dendritic cells obtained from the purified monocytes of three different donors. Two types of dendritic cells were compared, the maturation of which was induced either by TNF-α (200 U/ml) or by LCOS 1013 (25 μg/ml) (curve marked “LCOS”) for 48 hours. The horizontal axis corresponds to the number of irradiated dendritic cells deposited in each well, for a constant quantity of 10⁵ T lymphocytes per well. The proliferation of the T lymphocytes, stimulated by the dendritic cells, is determined after 5 days of culture by the incorporation of tritiated thymidine (vertical axis).

FIG. 8 shows the results of mixed allogenic and autologous lymphocytic reactions carried out with dendritic cells, the maturation of which was induced either by TNF-α (200 U/ml) or by LCOS 1013 at 5, 10 or 25 μg/ml (curve marked “LCO”) for 48 hours. The graphs were obtained in the same manner as the graphs shown in FIGS. 6 and 7, respectively.

All of the techniques of cell culture, cell labelling, phenotyping by fluorometry, etc., as well as the reagents (antibodies, tetanus toxin, . . . ) used in the experiments described in the following examples have been described in detail in an article by Karine Duperrier et al., Journal of Immunological Methods 238 (2000), p. 119-131.

### EXAMPLE 1

Process for the Production of Dendritic Cells from Human Monocytes With RU 41740

Peripheral blood is taken from the subject concerned. This blood is then centrifuged for 20 minutes at 200 g so as to diminish the contamination of the peripheral blood cells by platelets. The upper part containing most of the platelets and the plasma is carefully removed before the mononucleated cells are purified by centrifuging them on a Ficoll separation gradient, the density of which is 1.077. The layer of mononucleated cells is recovered, then washed twice with PBS buffer and deposited on a gradient containing 4 discontinuous densities of Percoll in order to isolate the monocytes. This gradient is constituted by concentrations of Percoll in isotonic solution of 75% (6.5 ml), 50.5% (15 ml), 40% (3.5 ml) and 30% (3 ml) in a Dulbecco medium without magnesium nor calcium and containing 5% human serum. 75 to 100 millions of cells are thus deposited in each tube and centrifuged at 1000 g during 25 minutes at 4° C. The low density cells, principally the monocytes, are harvested at the interface between the concentrations 40% and 50.5%, and washed twice with PBS. They are then resuspended in a culture medium, then deposited on culture plates containing 6 wells at a density of 5×10⁶ cells per well in a final volume of 3 ml and left to adhere for 1 hour at 37° C. This adhesion step can be replaced by an additional purification of the monocytes by means of a negative purification system using a mixture of monoclonal antibodies combining an anti-CD3, CD7, CD19, CD45A, CD56 and anti-IgG with the aid of a Macs type microbead system (Miltenyi Biotec). This additional purification makes it possible to obtain concentrations of monocytes having a purity higher than 90%.

The cells are then placed in culture in culture wells at 37° C. under 5% CO₂. The medium which consists of RPMI contains 200 IU/ml of recombinant human GM-CSF and 500 IU/ml of recombinant human IL-4 in a final volume of 6 ml. On day 3 and day 5, the cultures are renewed by removing 3 ml and adding 3 ml of fresh medium with the cytokines. On day 6 the cells are transferred to Teflon pots and cultivated to a density of 5×10⁵ cells per pot in 3 ml in the presence of RU 41740 at different concentrations or recombinant human TNF-α at a concentration of 200 IU/ml for 2 days. The cells are harvested on day 8, washed and cultivated in the absence of stimulation for a further 3 days.

At the end of the culture period, the quality of the maturation is assessed by measuring the expression of the molecules CD80, 83, 86, 14, 1a, HLA-DR at the surface of the cells. The labellings revealed that more than 80% of the viable monocytes have matured to dendritic cells characterized by the following phenotype: CD83+, CD86++, CD80++, HLA-DR+++, CD1a−, CD14−, CD40+++, CD54++, which places them in the group of highly differentiated tissue dendritic cells. FIG. No. 1 shows in fact that the molecules HLA-DR, HLA-DQ, CD40, CD54, CD80 and CD86 are present on the majority of the cells on D8, after addition of RU 41740 at 25 μg/ml, whereas the molecule CD14 is essentially no longer expressed. In order to study the action of RU 41740 on the maturation of the dendritic cells (DC), different concentrations of the molecule were tested and compared with the action of TNF.

The following 3 experiments (a, b, c) were carried out starting from monocytes of the same donor.

a) The concentrations of RU 41740 tested here are 0.1 μg/ml, 1 μg/ml and 10 μg/ml. Table 2 presents the results obtained with respect to the percentage of labelled cells and the mean fluorescence intensity (MFI).

TABLE 2
Percentage (%) of labelled cells and mean fluorescence intensity (MFI) of
the marker considered.

| | | | D6 | | | | | | | | | |
|---|---|---|---|---|---|---|---|---|---|---|---|---|
| | | | Undiffer- | | | | | | | | | |
| | | | entiated | | D8 | | | | | | | |
| | D0 | | dendritic | | RU 41740 | | RU 41740 | | RU 41740 | | TNF-alpha | |
| | Monocytes | | cells | | 0.1 μg/ml | | 1 μg/ml | | 10 μg/ml | | 200 IU/ml | |
| | % | MFI | % | MFI | % | MFI | % | MFI | % | MFI | % | MFI |
| HLA- | 98.9 | 275 | 98.3 | 552 | 98.7 | 978 | 99.1 | 1164 | 98.9 | 1195 | 98.9 | 1225 |
| DR | | | | | | | | | | | | |
| CD | 97.8 | 128 | 97.5 | 464 | 98.3 | 1340 | 98 | 1205 | 98.2 | 1328 | 98.4 | 1603 |
| 40 | | | | | | | | | | | | |
| CD | 98.6 | 41 | 95.6 | 85 | 96.4 | 220 | 96 | 210 | 95.6 | 282 | 97.4 | 295 |
| 54 | | | | | | | | | | | | |
| CD | 96.5 | 52 | 69.9 | 77 | 98.8 | 310 | 98.5 | 290 | 98 | 303 | 98.8 | 326 |
| 86 | | | | | | | | | | | | |
| CD | 0.2 | 325 | 5.5 | 119 | 82.1 | 156 | 76.1 | 165 | 84.7 | 184 | 84.5 | 173 |
| 83 | | | | | | | | | | | | |
| HLA- | 24.2 | 48 | 63.2 | 61 | 86.4 | 160 | 88.8 | 153 | 84.1 | 174 | 84.3 | 157 |
| DQ | | | | | | | | | | | | |
| CD | 0.1 | 46 | 3.1 | 112 | 60.5 | 173 | 50.1 | 187 | 70 | 250 | 62.1 | 129 |
| 80 | | | | | | | | | | | | |
| CD | 96.2 | 5395 | 1.9 | 199 | 1.55 | nd | 0.97 | nd | 1.06 | nd | 0.87 | nd |
| 14 | | | | | | | | | | | | |

These results are expressed with a margin of error of ±2.5%

After 8 days of culture, the expression of the CD14 molecule has disappeared (percentage less than 1.5%), whatever the conditions of culture.

The mean fluorescence intensity (MFI) of the labellings of the molecules HLA-DR, CD40, CD54 and CD86 appears weaker in the presence of the doses of RU 41740 used than in the presence of TNF-α.

On the other hand, the molecules CD83 and HLA-DQ are expressed slightly more in the presence of a concentration of RU 41740 of 10 μg/ml than with TNF-α.

Furthermore, the MFI of the CD80 molecule always appears higher in the presence of RU 41740.

According to these results, it can be seen that RU 41740 induces the maturation of the dendritic cells, even at low concentrations. The MFI of the different markers however appears to be increased for the highest concentrations of RU 41740 tested, and approaches the results obtained in the presence of TNF-α.

The action of RU 41740 on the dendritic cells at even higher concentrations was hence investigated in a second series of experiments.

b) The concentrations of RU 41740 tested in this case are 5 μg/ml, 10 μg/ml and 50 μg/ml.

The results are presented in Table No. 3.

The MFI of the different molecules of adhesion, co-stimulation and class II HLA molecules appears to be increased in a manner dependent on the concentration of RU 41740 added to the culture medium. The CD40 molecule always exhibits a lower MFI in the presence of RU 41740 compared to the concentration obtained in the presence of TNF-alpha.

CD83, which is the specific marker of maturation, has a percentage and an intensity of fluorescence which also increase as a function of the quantity of RU 41740. The molecule HLA-DQ exhibits a maximal expression at 10 μg/ml of RU 41740.

TABLE No. 3
Percentage (%) of labelled cells and mean fluorescence intensity
(MFI) of this marker (nd = could not be determined)

| | | | D6 | | | | | | | | | |
|---|---|---|---|---|---|---|---|---|---|---|---|---|
| | | | Undiffer- | | | | | | | | | |
| | | | entiated | | D8 | | | | | | | |
| | D0 | | dendritic | | RU 41740 | | RU 41740 | | RU 41740 | | TNF-alpha | |
| | Monocytes | | cells | | 5 μg/ml | | 10 μg/ml | | 50 μg/ml | | 200 IU/ml | |
| | % | MFI | % | MFI | % | MFI | % | MFI | % | MFI | % | MFI |
| HLA- | 99.2 | 275 | 98.3 | 552 | 98.9 | 1207 | 99.1 | nd | 99.3 | nd | 99.1 | 946 |
| DR | | | | | | | | | | | | |
| CD | 97.8 | 128 | 97.5 | 464 | 99.8 | 1417 | 99.4 | 1441 | 99.8 | 1507 | 99.6 | 1728 |
| 40 | | | | | | | | | | | | |
| CD | 98.6 | 41 | 95.6 | 85 | 97.1 | 195 | 97 | 216 | 98.2 | 255 | 95.6 | 255 |
| 54 | | | | | | | | | | | | |
| CD | 96.5 | 52 | 69.9 | 77 | 98.9 | 267 | 98.7 | 271 | 99.7 | 248 | 99.5 | 273 |
| 86 | | | | | | | | | | | | |
| CD | 0.2 | 325 | 5.5 | 119 | 67.9 | 132 | 74.6 | 147 | 84.8 | 159 | 89.5 | 196 |
| 83 | | | | | | | | | | | | |
| HLA- | 24.2 | 48 | 63.2 | 61 | 68.5 | 189 | 85.9 | 191 | 80.3 | 143 | 84.2 | 183 |
| DQ | | | | | | | | | | | | |
| CD | 0.1 | 46 | 3.1 | 112 | 63 | 86.7 | 64.6 | 101 | 76.1 | 95.8 | 70.5 | 93.5 |
| 80 | | | | | | | | | | | | |
| CD | 96.2 | 5395 | 1.9 | 199 | 0.47 | nc | 1.1 | n | 0.66 | nd | 0.61 | nd |
| 14 | | | | | | | | | | | | |
| CD | | | | | 2.52 | nd | 1.55 | nd | 2.9 | nd | 0.21 | nd |
| 1a | | | | | | | | | | | | |

These results comprise a margin of error of ±2.5%.

With a higher concentration (50 pg/ml) of RU 41740 compared to the experiment in Table 2, the MFI of the molecules CD40 and CD54 has increased, whereas the MFI of other molecules has diminished (molecules CD86 and HLA-DQ). Some markers, like CD80 and CD54, are expressed with the same intensity after a maturation with 50 μg/ml of RU 41740 or with TNF-α.

Concentrations of RU 41740 both higher and lower than 50 μg/ml were then tested in a third series of experiments.

c) The concentrations of RU 41740 tested are 25 μg/ml, 50 μg/ml and 100 μg/ml.

The results are presented in Table No. 4.

TABLE No. 4
Percentage (%) of labelled cells and mean fluorescence intensity
(MFI) of this marker (nd = could not be determined).

| | | | D6 | | | | | | | | | |
|---|---|---|---|---|---|---|---|---|---|---|---|---|
| | | | Undiffer- | | | | | | | | | |
| | | | entiated | | D8 | | | | | | | |
| | D0 | | dendritic | | RU 41740 | | RU 41740 | | RU 41740 | | TNF-alpha | |
| | Monocytes | | cells | | 25 μg/ml | | 50 μg/ml | | 100 μg/ml | | 200 IU/ml | |
| | % | MFI | % | MFI | % | MFI | % | MFI | % | MFI | % | MFI |
| HLA- | 99.2 | 275 | 98.3 | 552 | 98.5 | 630 | 97.5 | 823 | 99.4 | 682 | 99.4 | 694 |
| DR | | | | | | | | | | | | |
| CD | 97.8 | 128 | 97.5 | 464 | 99.7 | 1592 | 99.1 | 1958 | 99.8 | 2088 | 99.3 | 1480 |
| 40 | | | | | | | | | | | | |
| CD | 98.6 | 41 | 95.6 | 85 | 98.5 | 280 | 97.2 | 321 | 98.9 | 361 | 98.2 | 249 |
| 54 | | | | | | | | | | | | |
| CD | 96.5 | 52 | 69.9 | 77 | 99.3 | 284 | 98.3 | 291 | 99.6 | 295 | 99.5 | 284 |
| 86 | | | | | | | | | | | | |
| CD | 0.2 | 325 | 5.5 | 119 | 86.6 | 192 | 91.2 | 227 | 92.8 | 213 | 89 | 166 |
| 83 | | | | | | | | | | | | |
| HLA- | 24.2 | 48 | 63.2 | 61 | 79 | 109 | 84.8 | 123 | 78.8 | 118 | nd | nd |
| DQ | | | | | | | | | | | | |
| CD | 0.1 | 46 | 3.1 | 112 | 74 | 128 | 82.4 | 165 | 90.8 | 196 | nd | nd |
| 80 | | | | | | | | | | | | |
| CD | 96.2 | 5395 | 1.9 | 199 | 1.03 | nd | 4.07 | nd | 1.75 | nd | 3.27 | nd |
| 14 | | | | | | | | | | | | |

These results comprise a margin of error of ±2.5%.

The molecules CD40, CD54, CD86 and HLA-DR are present on more than 97% of the cells at the three concentrations of RU 41740. Their MFI are comparable to those obtained with TNF-α for a concentration of 25 μg/ml of RU 41740. However, in this experiment they continue to increase with higher concentrations of RU 41740.

CD83 is present on a very high percentage of cells at 100 μg/ml and at 50 μg/ml, higher than the results obtained with TNF-α. Its MFI appears to be higher with 50 μg/ml.

At 100 μg/ml of RU 41740, the fluorescence intensities of the class II HLA molecules tend to diminish, and this may suggest that this concentration is the limit concentration of RU 41740.

All of the experiments suggest that the optimal dose of RU 41740 is situated between 10 and 50 pg/ml.

### EXAMPLE 2

Process for the Production of Dendritic Cells from Human Hematopoietic CD34+ Stem Cells With RU 41740

Experimental Conditions:

Purification: After Ficoll, the CD34+ represented 4.2% of the mononucleated cells (MNC).

After purification by positive selection with the MAC System procedure (Miltenyi Biotec) with beads coated with an anti-CD34 antibody, 90% of the cells are CD34+.

Placing in Culture:

Culture Medium: RPMI containing 10% of FCS, 2% of penicillin/streptomycin, 1% of glutamine and 1% of sodium bicarbonate.

Culture in 2 ml wells with 5×10⁵ cells per well.

Cytokines Present in the Culture Medium:

from D0 to D5: FCS at 25 ng/ml, GM-CSF at 100 ng/ml and

either TNF-α at 2.5 ng/ml

or RU 41740 at 6.25 ng/ml

from D5 to D14: GM-CSF at 100 ng/ml

Duplication of the Cultures: -the cells are homogenized in the well, then 1 ml sample is taken and deposited in a fresh well. 1 ml of culture medium is then added to the fresh well and the old one, as well as GM-CSF as a function of the medium added.

Markers: CD34-CD45-CD14-HLA-DR-CD40-CD54-CD83-CD86-CD1a.

A fluorescence-labelled anti-KLH primary antibody was used as control in order to be able to subtract the non-specific fluorescence of the signals obtained.

Results:

TABLE No 5
Percentage of cells expressing a cell marker at different
times of culture

| | D8 | | D12 | | D14 | |
|---|---|---|---|---|---|---|
| | TNF-α | RU 41740 | TNF-α | RU 41740 | TNF-α | RU 41740 |
| CD14 | 40 | 37 | 63.2 | 47.4 | 54.1 | 52.5 |
| CD34 | 0.7 | 17 | | | | |
| CD45 | 96 | 96 | | | | |
| CD83 | 0.5 | 0.2 | 1.3 | 1.3 | 0.8 | 1 |
| CD86 | 13 | 11 | 7.4 | 10.8 | 2.2 | 4.7 |
| CD1a | 8.7 | 17 | 8.4 | 17.5 | 10.3 | 28.9 |
| CD40 | 44 | 38 | 64.3 | 59.4 | | |
| CD54 | 33 | 56 | 61.8 | 57.9 | | |
| HLA- | 88 | 80 | 80.3 | 65.3 | 69.9 | 69.54 |
| DR | | | | | | |
| HLA- | | | 54.1 | 52.3 | | |
| DQ | | | | | | |
| CD80 | | | 5.3 | 16 | | |

CD1a is expressed more with RU 41740 (17.5% of cells on D12) than with TNF-α, unlike CD14, which is expressed more with TNF-α at the cell surface.

The levels of expression in the presence of RU 41740 of CD40, CD54 or HLA-DR are essentially identical to the levels of expression in the presence of TNF-α.

CD83 is not expressed whatever the conditions.

### EXAMPLE 3

Pr c ss for the Production of Dendritic c lls Starting From CD34+ Cells of Human Cord Blood With RU 41740

The experimental conditions are similar to Example 2, except the fact that the cells are derived from cord blood:

Purification: After Ficoll, the CD34+ cells represent 0.83% of the mononucleated cells (MNC).

After purification by positive selection, 18.1% of CD34+ are obtained.

2×10⁶ cells were obtained.

Duplication: on D6, D7,D8, D10 and D12.

TABLE No. 6

| | D7 | | D9 | | D10 | | D12 | | D13 | | D14 | |
|---|---|---|---|---|---|---|---|---|---|---|---|---|
| | TNF-α | RU 41740 | | RU | | RU | | RU | | RU | | RU |
| | (200 IU/ml | (10 μg/ml) | TNF-α | 41740 | TNF-α | 41740 | TNF-α | 41740 | TNF-α | 41740 | TNF-α | 41740 |
| CD14 | 32 | 20.39 | 59.6 | 43.52 | 60 | 50.05 | 66.77 | 59.23 | 57.73 | 52.28 | 46.77 | 30.88 |
| CD34 | 19.79 | 19.92 | 2.53 | 2.94 | 2.26 | 2.08 | 0.69 | 1.25 | 0.45 | 0.61 | 0.38 | 0.57 |
| CD45 | 99.84 | 97.57 | 99.96 | 99.76 | 99.97 | 99.86 | 99.85 | 99.92 | 99.69 | 99.82 | 99.93 | 99.91 |
| CD16 | 2.24 | 3.68 | 2.16 | 2.78 | 2.11 | 4.89 | | | | | | |
| CD83 | 1.09 | 0.39 | 4.24 | 4.9 | 2.46 | 1.19 | 0.71 | 1.35 | 3.19 | 3.41 | 1.22 | 0.83 |
| CD86 | 11.61 | 11.97 | 22.23 | 13.69 | 9.23 | 7.48 | 13.94 | 11.91 | 12.78 | 17.1 | 19.33 | 10.56 |
| CD1a | 4.41 | 13.95 | 23.75 | 20.32 | 20.63 | 22.68 | 27.9 | 20.14 | 36.13 | 23.67 | 34.2 | 16.63 |
| CD40 | | | | | | | 75.91 | 55.23 | 63.35 | 56.02 | 64.56 | 41.47 |
| CD54 | | | | | | | 74.66 | 65.62 | 61.7 | 58.57 | 64.4 | 41.69 |
| HLA-DR | | | | | | | 81.34 | 73.8 | 73.04 | 63.28 | 57.84 | 37.69 |

Percentage of Labelled Cells

CD34+ cells of cord blood placed in culture until D6 with GM-CSF, SCF and RU 41740 (10 μg/ml) or TNF-α (200 IU/ml), then from D6 to D14 with GM-CSF.

In the two culture protocols, the changes in the expression of the surface molecules show the same tendencies:

increase of CD14 up to D12, then diminution

diminution then disappearance of the molecule CD34 on D12

very weak expression of CD83 and CD16 which remains stable.

CD1a is expressed early at D7 on the cells treated with RU 41740 then stabilizes around 23% at D14, thus revealing more rapid kinetics with RU 41740 than with TNF-α.

After D12, the molecules CD40, CD54 and HLA-DR are expressed to a lesser extent in the presence of RU 41740 than in the presence of TNF-α.

### EXAMPLE 4

Process of Differentiation of Langerhans Cells

The stem cells are obtained after a passage of cord blood through a Ficoll gradient. The mononucleated CD34+ cells are purified by positive selection with an anti-CD34 monoclonal antibody (Immu 133.3, Immunotech Marseille, France). After purification, more than 90% of the cells are CD34+.

These CD34+ cells are then cultivated in the presence of GM-CSF (100 ng/ml) and TNF-α (2.5 ng/ml) or RU 41740 (10 μg/ml) for 12 days in a medium: RPMI-FCS 10%—penicillin streptomycin 2%, glutamine 1%—sodium bicarbonate 1 % and 10 mM of HEPES. At the end of the culture period at D12, the cells are labelled with the anti-CD1a, CD14, Lag, E-cadherin, DR and DQ antibodies so as to identify the Langerhans cells which are Lag+, CD1a+, CD14−, DR+ and DQ+.

The results presented in Table No. 7 show a better efficacy of RU 41740 than TNF-α in the differentiation of the Langerhans cells.

TABLE 7

| | TNF-α | RU 41740 |
|---|---|---|
| CD1a+ DR+ | 8% | 17% |
| Lag+ DR+ | 5.2% | 8.6% |

### EXAMPLE 5

Process for the Production of Mature Dendritic Cells From the Mononucleated Cells of the Dog With RU 41740.

An elutriator is an apparatus which makes it possible to subject cells to two opposing forces, one centrifugal and the other centripetal, in a liquid medium. This makes it possible to separate the cells according to their size and their density, while maintaining them in their physiological medium. This procedure is particularly advantageous for separating the dog cells (monocytes/lymphocytes) which form aggregates in gradients of the Ficoll type.

Elutriation Medium:

PBS 1X-FCS 2%-EDTA 0.01%

Preparation of the Mononucleated Cells:

The pouch of blood is collected on CPD (citrate phosphate dextrose), the blood is diluted with sodium chloride, centrifuged at 800 rev./min for 15 min without braking in order to remove as many platelets as possible.

A Ficoll is carried out at 1600 rev./min for 25 min without braking.

The MNC are recovered and washed twice with PBS (the first to remove platelets). The cell concentration is 5×10⁶cells/ml in PBS or in the elutriation medium.

Protocol for Obtaining Monocytes

Flow rate: 25 ml/min (control with the pump according to the calibration line)

Vary the Speed of Centrifugation:

Loading of the cells at 3200 rev./min in 300 ml

3000 rev/min in 250 ml

2700 rev/min in 200 ml

2500 rev/min in 200 ml

2300 rev/min in 200 ml

2100 rev/min in 200 ml

Rotor off in 200 ml

Results

The fraction obtained at 2700 rev/min contains monocytes with a purity higher than 80%.

Culture

The dog monocytes are cultivated in the same manner as human monocytes and with the same human differentiation factors: GM-CSF, TNF or RU 41740. On the other hand, IL4 is specific for the canine species. The dendritic cells thus obtained possess the same morphology as dendrites, but the surface markers are not comparable to those of man although they are CD14−, DR+ and DQ+, because we do not have available specific antibodies in this species. RU 41740 has the same effects as TNF-α.

### EXAMPLE 6

Use of Mature Dendritic Cells in the Emergence of an Anti-Tumor Response.

Thyrocalcitonin is a relatively weak immunogenic tumor antigen expressed strongly in medullary cancers of the thyroid. In the system used, the inventors have been able to reproduce T lymphocytic clones directed against thyrocalcitonin. They are cytotoxic clones capable of having an anti-tumor activity in the cancers caused by thyrocalcitonin.

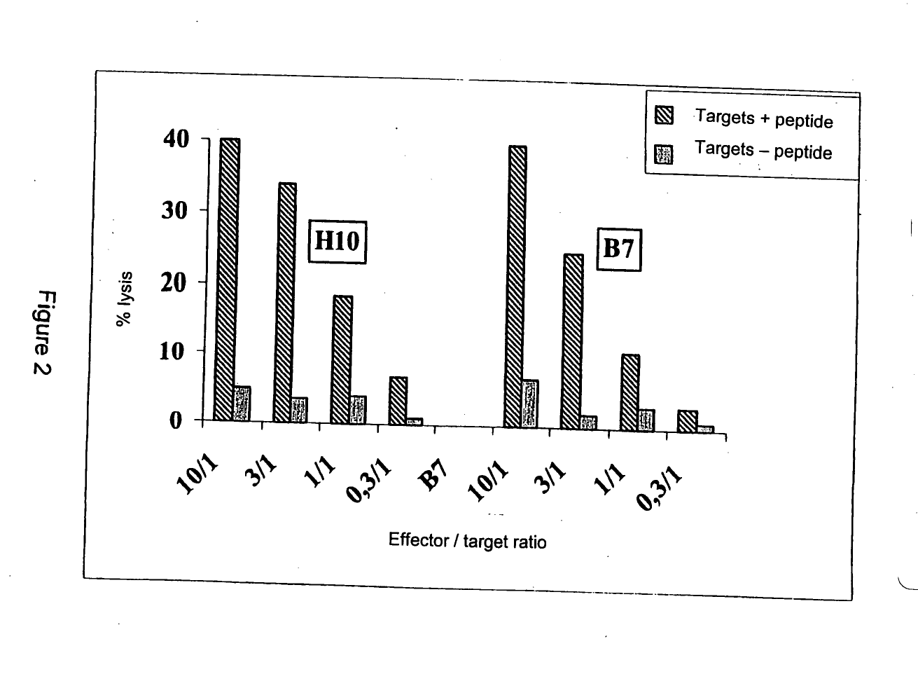

FIG. 2 illustrates the generation of cytotoxic T lines specific for the thyrocalcitonin peptide.

Dendritic cells derived from monocytes after culture in the presence of GM-CSF, IL-4 and RU 41740 are incubated with the thyrocalcitonin peptide, then placed in culture in the presence of autologous T lymphocytes in order to induce the activation of T lymphocytes specific for the peptide.

After several stimulations of the T lymphocytes with the aid of dendritic cells then with EBV cells incubated with the peptide, lines of cytotoxic T cells (H10 and B7) capable of lysing specifically target EBV cells incubated with the thyrocalcitonin peptide could be generated.

### EXAMPLE 7

Use of Mature DC in Anti-Infectious Activities

By using the tetanus anatoxin as antigen presented by the MODC (Monocyte Dendritic Cells), it was possible to generate anti-tetanus anatoxin cytotoxic lines, which quite obviously demonstrates that this system makes it possible to have a primary immunization towards antigens of the infectious type.

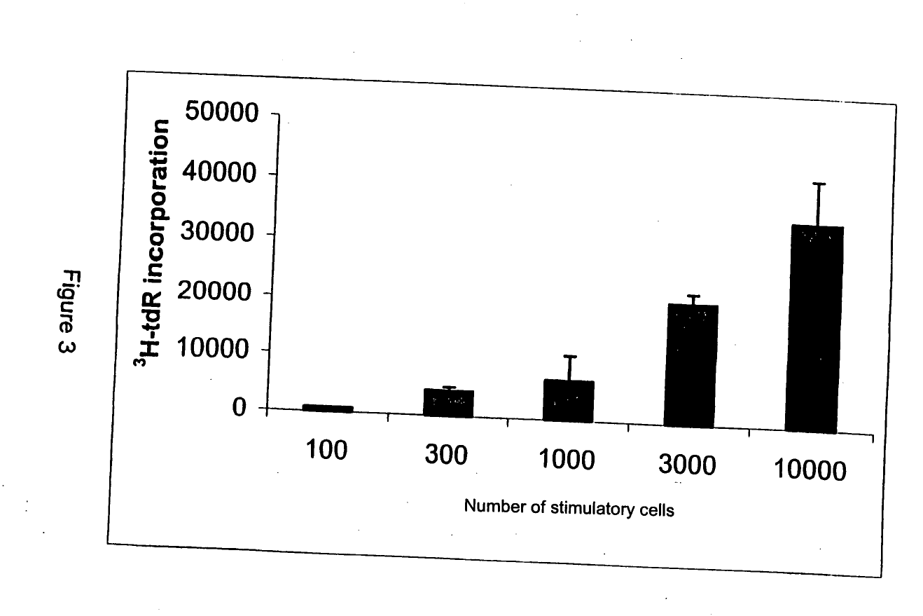

FIG. 3 represents a secondary response of the anti-tetanus anatoxin lines by the dendritic cells derived from human monocytes cultivated in the presence of GM-CSF, IL-4 and RU 41740

### EXAMPLE 8

Use of Mature Dendritic Cells in the Induction of a Tolerance Response.

The dendritic cells can cause an anergic reaction under certain culture conditions. The culture of these cells in the presence of immunosuppressants like cyclosporin or histamine leads to a modification of the membrane antigens which will induce a tolerance response and not a cytotoxic response. In this manner, it is hence possible to educate the dendritic cells in order to direct them towards a tolerance reaction. We give as an example the influence of cyclosporin A (CsA) on the maturation of the dendritic cells.

Purified monocytes were cultivated in the presence of GM-CSF and IL-4 for 6 days and in the presence of RU 41740 for 2 days more, in a medium containing 10% AB type human serum. 1 μg/ml or 5 μg/ml of CsA were or were not added right at the start of culture. The expression of the molecules HLA-DR, CD83, CD86, CD80, CD40 and CD1a was analyzed by flow cytometry (Table No 8).

TABLE No 8

| | Without CsA | | CsA (1 μg/ml) | | CsA (5 μg/ml) | |
|---|---|---|---|---|---|---|
| | % ± SD | MFI | % ± SD | MFI | % ± SD | MFI |
| HLA- | 99 ± 0.8 | 1605 | 98.8 ± 0.9 | 1504 | 99.2 ± 0.7 | 1101 |
| DR | | | | | | |
| CD40 | 98.9 ± 0.6 | 1697 | 98.4 ± 0.8 | 1314 | 99 ± 0.6 | 1278 |
| CD86 | 97.7 ± 1.8 | 373 | 98.6 ± 1.2 | 318 | 96.4 ± 3.3 | 253 |
| CD83 | 77.1 ± 8.5 | 169 | 64.2 ± 20 | 170 | 49.9 ± 6.2 | 132 |
| CD80 | 78.1 ± 5.6 | 216 | 68.9 ± 2.6 | 162 | 58.3 ± 17 | 143 |
| CD1a | 14.3 ± 9.5 | 45.5 | 27 ± 7 | 74 | 25.9 ± 6.5 | 78 |

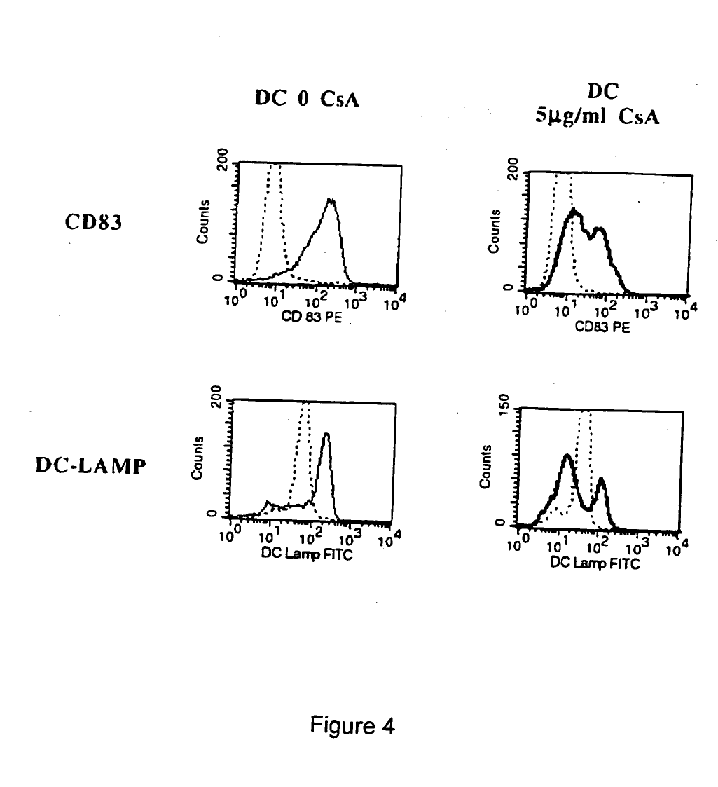

This study demonstrates that cyclosporin causes a marked diminution of the expression of the molecules CD83 and CD80 as well as an increase of the expression of CD1a. A graphic analysis (FIG. 4) reveals in reality the existence of two cell populations, one CD83+ and the other CD83− which has immunoregulatory properties.

Indeed the dendritic cells in the presence of CsA (CsA-MODC) are capable of directing the response of the T lymphocytes towards a type TH2 response which promotes a common suppressive reaction.

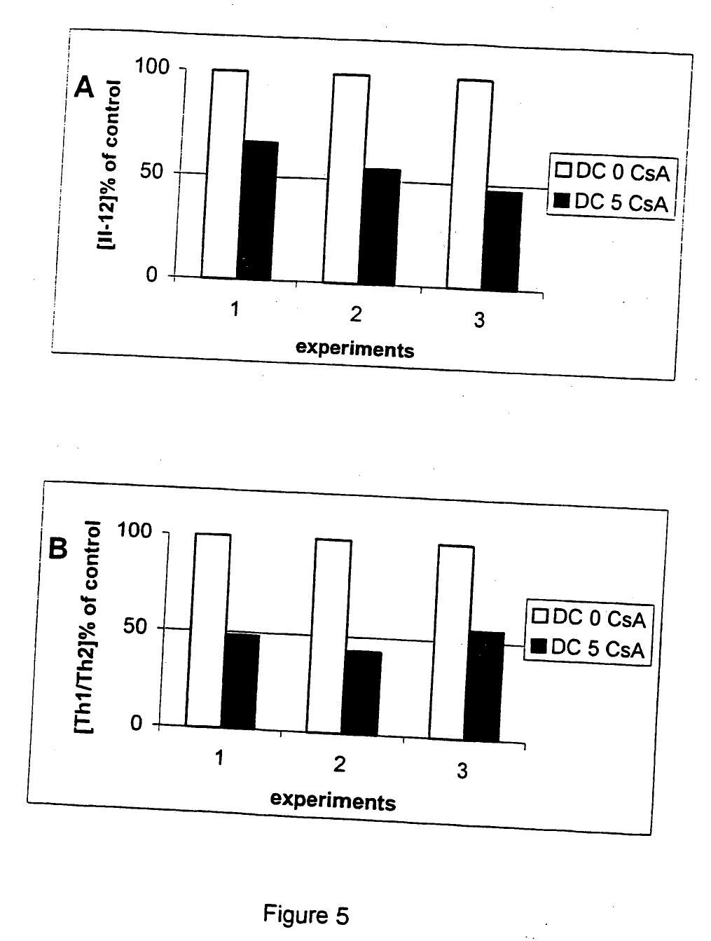

FIG. 5 illustrates this polarization of the immune response towards a type Th2 response by dendritic cells derived from monocytes and treated with cyclosporin A.

Dendritic cells derived from monocytes cultivated in the presence of GM-CSF, IL-4 and RU 41740 and treated with cyclosporin A secrete less IL-12 than the untreated dendritic cells (FIG. 5A).

Furthermore, the ratio of the secretion of cytokines IFN-γ/1-10 (Th1/Th2) by T cells is diminished when the T cells are stimulated by dendritic cells treated with cyclosporin (FIG. 5B).

### EXAMPLE 9

Use of Mature DC to Induce a Mixed Allogenic or Autologous Lymphocytic Culture.

In the presence of mature MODC, the allogenic T lymphocytes, on day 6 and even on day 8 have an extremely extensive capacity to proliferate, very much higher than that which is observed with the use of the allogenic T lymphocytes and allogenic monocytes. It is the same for a mixed autologous culture.

Mixed autologous lymphocytic cultures were hence induced by cells derived from monocytes (MODCs) generated in the presence of GM-CSF (200 IU/ml, IL-4 (500 IU/ml and RU 41740 (10 μg/ml).

Ten thousand MODCs, irradiated at 30 Grays, are cultivated with different lymphocytic subpopulations selected either by means of Dynal magnetic beads (CD4+ and CD8+) or with the select module of the FACS Calibur (CD8bright, CD28− and CD8bright, CD28+ autos). In Table 8, the values indicated represent the incorporation of tritiated thymidine after mixed autologous culture, except(**). The experiments are carried out in triplicate, except (*), in duplicate.

TABLE No. 9

| | | | | Student test |
|---|---|---|---|---|
| | | | | compared with |
| | | | | negative control |
| Condition | Values | | | P = |
| (100.000 CD4+ autos) + | 7898 | 10720 | 8613 | |
| (35.000 CD4+ autos) | | | | |
| (100.000 CD4+ allos) + | 119066 | 193392 | 206319 | 3.827E−03 |
| (35.000 CD4+ allos) (**) | | | | |
| (100.000 CD4+ autos) + | 37808 | 45223 | 42027 | 1.458E−04 |
| (35.000 CD28− autos) | | | | |
| (100.000 CD4+ autos) + | 32990 | 54800 | | 2.394E−02 |
| (35.000 CD28+ autos) | | | | |
| (100.000 CD4+ autos) + | 13985 | 12941 | 13573 | 3.978E−03 |
| (100.000 CD8+ autos) | | | | |
| (*) | | | | |

| Condition | Mean | Standard deviation |
|---|---|---|
| (100.000 CD4+ autos) + | 9077 | 692 |
| (35.000 CD4+ autos) | | |
| (100.000 CD4+ allos) + | 172926 | 22198 |
| (35.000 CD4+ allos) (**) | | |
| (100.000 CD4+ autos) + | 41686 | 1753 |
| (35.000 CD8bright 28− | | |
| autos) | | |
| (100.000 CD4+ autos) + | 43895 | 7711 |
| (35.000 CD8bright | | |
| CD28+ autos) (*) | | |
| (100.000 CD4+ autos) + | 13500 | 248 |
| (100.000 CD8+ autos) | | |

### EXAMPLE 10

Processes for the Production of LCOS 1013 and LCOS 1014.

A. General Presentation of LCOS 1013

LCOS 1013 is an analogue of RU 41740 consisting of a set of substances extracted from a culture lysate of Klebsiella pneumoniae. Its mode of production is based on the sequence of steps or stages, the general scheme of which is presented below. Media usable for the cultures as well as a summary presentation of how each step proceeds are described in more detail after this diagram.

The production technique described below, purely as a guide, corresponds to an operational unit based on the volume of bacterial culture obtained in a 600 litres fermenter.

STAGE 1: Preparation of the inoculum
↓
STAGE 2: Preculture
↓
STAGE 3: Culture in fermenter
↓
STAGE 4: End of culture and addition of lysing agents
↓
STAGE 5: Lysis
↓
STAGE 6: Molecular sieving with concentration
↓
STAGE 7: Lyophilization of the lysate
↓
STAGE 8: Extraction with acetone
↓
STAGE 9: Extraction with methanol
↓
STAGE 10: Redissolution
↓
STAGE 11: Centrifugation
↓
STAGE 12: Ultracentrifugation
↓
STAGE 13: Filtration
↓
STAGE 14: Lyophilization
↓
STAGE 15: Mixing

B. Description of Different Media Usable for the Culture of Bacteria

The culture of bacteria with a view to producing LCOS 1013 following the sequence of steps specified above can be carried out by using the culture media containing the following constituents:

Nutritive Broth

| | | | |
|---|---|---|---|
| Nutritive broth | | | |
| Papaïn soya peptone | | 4 ± 2 | g/l |
| Yeast autolysate | | 4 ± 2 | g/l |
| Sodium chloride | | 5 ± 2 | g/l |
| Sodium hydroxide | qsp for pH | 7.4 ± 0.2 | |
| Roux dish | | | |
| Sodium chloride | | 5 ± 2 | g/l |
| Anhydrous glucose | | 5 ± 2 | g/l |
| Yeast autolysate | | 12 ± 3 | g/l |
| Papaïn soya peptone | | 5 ± 2 | g/l |
| Gelose | | 30 ± 4 | g/l |
| Sodium hydroxide | qsp for pH | 7.5 ± 0.2 | |
| Dipotasium phosphate | | 4 ± 2 | g/l |
| Monopotassium phosphate | | 0.5 ± 0.9 | g/l |
| Preculture medium | | | |
| Papaïn soya peptone | | 20 ± 3 | g/l |
| Yeast autolysate | | 10 ± 2 | g/l |
| Sodium chloride | | 5 ± 2 | g/l |
| Dipotasium phosphate | | 3.5 ± 1 | g/l |
| Monopotassium phosphate | | 1 to 2 | g/l |
| Culture medium for fermenter | | | |
| Autolysate of baker's yeast | | 10 ± 2 | g/l |
| Sodium chloride | | 5 ± 2 | g/l |
| Papaïn soya peptone | | 20 ± 3 | g/l |
| Dipotasium phosphate | | 3.5 ± 1 | g/l |
| Monopotassium phosphate | | 1 to 2 | g/l |

C. Detailed Description of the Different Stages of Production of LCOS 1013

Stage 1: Preparation of the Inoculum

The objective of this stage is to carry out, on gelosed medium in a Roux dish, the culture of Klebsiella pneumoniae necessary for the inoculation of a preculture in liquid medium, starting from a cryotube or a lyophilizate derived from the work bank. The work bank is carried out for example starting from a strain of Klebsiella pneumoniae of the Pasteur Institute reference CIP 52.145

For that, the strain is revived by preparing a bacterial suspension in nutrient broth, starting from which Roux dishes are inoculated, which are then incubated at 37° C.±0.5 for 20 to 24 hours.

Stage 2: Precultures

Starting from the inoculum prepared at stage 1, a preculture is prepared in a 3 litres bottle, which will be used to inoculate a 35 1 fermenter, which in turn will be used in a second stage to inoculate a 600 l fermenter.

The first preculture is prepared in the preculture medium described above, to which is added a sterile solution of glucose containing 30 g of glucose for 3 litres. The second preculture, of 35 litres, is prepared in the culture medium for the fermenter described above, to which 400 g of glucose are added.

Satellite bottles containing respectively a sterile solution of 10 N sodium hydroxide, a sterile solution of orthophosphoric acid diluted to ½ and a sterile anti-foam solution, can be used for the second preculture, in the fermenter, in order in particular to maintain the pH around 6.5 throughout the entire preculture, lasting namely for about 5 hours at about 37° C., with shaking.

Stage 3: Culture in Fermenter

The objective of this step is to produce under defined conditions in the fermenter a bacterial culture containing 1.7×10¹⁰ or more bacteria/ml in order to make possible the subsequent extraction of a satisfactory quantity of product.

For that, the suspension of bacteria contained in the 35 l fermenter is transferred to a 600 litres fermenter containing the culture medium for the fermenter described above, to which 5 kg of glucose were added.

As in the case of the 35 litres of preculture, satellite vats containing respectively a sterile solution of 10 N sodium hydroxide and a sterile solution of orthophosphoric acid diluted to ½ are used for the adjustment of the pH around 6.5 as well as a satellite vat containing a sterile anti-foam solution. The culture is grown during about 7 hours at 37° C.±1° C., with shaking.

Stage 4: End of Culture and Addition of the Lysing Agents

The objective of this stage is to inactivate the culture by the action of lysing agents and by heating from 55 to 75° C. for a time equal to or exceeding 40 minutes. For that the following lysing agents are used:

Sterile solution of polysorbate 80,

Sterile solution of EDTA,

Sterile solution of lysozyme hydrochloride.

At the end of culture, the pH is brought to 5.8±0.3 with the aid of a sterile solution of orthophosphoric acid, then the above lysing agents are transferred to the culture.

The temperature of the content of the fermenter is then brought to 65° C.±10° C. and maintained at this temperature with shaking for at least 40 minutes.

The biolysate obtained is then transferred to an industrial lysis vat (preheated to 65° C.±10° C.) and maintained at this temperature for at least 20 minutes, before being brought to 37° C.±2° C.

Stage 5: Lysis

The lysis step consists of rupturing the cell wall of the bacteria enzymatically, and this leads to the release of the cytoplasmic constituents of the microbial cells. It is carried out by maintaining the prelysed suspension of the preceding stage at 37° C.±2° C. in the lysis vat for at least 6 days.

Stage 6: Molecular Sieving and Concentration

In this step the volume of crude lysate is reduced by a half in order to diminish the amount to be treated in the following stages. The concentration is carried out for example on an ultrafilter equipped with a filtering cartridge of the CARBOSEP type with a ≦100 KD cut-off threshold, thus removing unbound small molecules.

Stage 7: Lyophilization of the Lysate

In order to obtain the crude lysate derived from stage 6 in solid form making possible subsequent extractions with solvents, a lyophilization operation is carried out immediately after the concentration stage. A brown powder, sticky to the touch, is thus obtained.

Stage 8: Extraction With Acetone:

The lyophilized lysate of LCOS 1013, derived from Stage 7, contains lipids that are partially removed by solid-liquid extraction, using acetone at room temperature.

The volume of acetone used is proportional to the mass of the lyophilisate. This volume is obtained according to the following calculation:

10×mass to be extracted≦V solvent≦20×mass to be extracted, with preferably (V solvent/mass to be extracted)=15

The product is recovered by centrifugation, then dried.

Stage 9: Extraction With Methanol

After extraction with acetone, the product obtained in stage 8 still contains residual lipids and pigments which are removed by solid-liquid extraction using methanol at room temperature.

The volume of methanol used is proportional to the mass of the lyophilisate.

This volume is obtained according to the following calculation:

10×mass to be extracted≦V solvent≦20×mass to be extracted, with preferably (V solvent/mass to be extracted)=15

The product is recovered by centrifugation, then dried.

Stage 10: Redissolution

“The methanol extract” obtained at stage 9 is redissolved in aqueous solution in order to make possible the subsequent isolation of the product by centrifugation and ultrafiltration.

Stage 11: Centrifugation

The crude extract in suspension derived from stage 10 contains denatured proteins and materials insoluble in water which are removed by centrifugation between 14000 and 18000 g.

The centrifuged product has a slightly colloidal appearance and is beige brown in colour.

Stage 12: Ultrafiltration

This stage represents the essential step in the isolation of the product.

The product derived from stage 11 contains substances of different molecular sizes: mineral salts, proteins, glycoproteins, etc . . .

The macromolecules of MW≧300000 constituting the product are isolated by ultrafiltration through a membrane with a cut-off threshold of 300 KD.

The product, once introduced into the interior of the apparatus is circulated continually by means of a pump in a closed circuit comprising an ultrafilter. The medium is maintained homogeneous by means of shaking. The pressure created by the pump on the membrane filter compels a part of the solute to pass through this membrane (permeate).

The part retained (retentate) remains in circulation. Since the operation proceeds at constant volume, the loss of volume is compensated continuously by the supply of the same volume of water.

At the end of the operation the solution is ultrafiltered. The ultrafiltrate obtained is a slightly yellow translucent liquid solution.

Stage 13: Filtration

The clarification of the centrifugation supernatant obtained in stage 11 is improved by filtration through a membrane with nominal retention of 1.2 μm.

In this way an opalescent “solution of glycoproteins”, light beige to cream in colour, is obtained.

Stage 14: Lyophilization

In order to ensure that the product is well preserved, the solution derived from stage 13 is then lyophilized by a procedure comprising a freezing step, then the lyophilization properly so called.

The lyophilized glycoproteins have the appearance of a flocculent, creamy white, hygroscopic powder.

Stage 15: Mixing

The objective of this last step is to homogenize the lyophilized glycoproteins derived from stage 14.

Spraying may advantageously replace lyophilization and mixing (LCOS 1014)

### EXAMPLE 11

Study of the Effect of the Molecule LCOS 1013 on the Maturation of the Dendritic Cells Derived From Human Monocytes.

The effect of LCOS 1013 on the maturation of the dendritic cells generated from human monocytes was studied on cells derived from 3 different donors, by comparison with TNF-α.

A. Protocol

The purified monocytes (more than 90%) were placed in culture in RPMI medium containing 10% AB human serum in the presence of growth factors GM-CSF (200 U/ml) and IL-4 (500 U/ml) for 6 days in order to induce the differentiation of the monocytes into immature dendritic cells. The maturation of the cells was induced by addition of TNF-α (200 U/ml) or the compound LCOS 1013 (25 μg/ml) for 48 hours.

The phenotype of the cells thus generated (after 8 days of culture) was examined by flow cytometry. The expression of the marker specific for maturation of the dendritic cells (CD83), of the co-stimulatory molecules CD80, CD86, CD40, of the adhesion molecule CD54, of the class II HLA-DR molecule of the major histocompatibility complex was analyzed. The expression of the CD1a molecule, specific marker of the Langerhans cells, immature dendritic cells, was also tested.

The functional properties of the cells were studied with the aid of mixed lymphocytic reactions (MLR). In order to do this, a range of concentrations of dendritic cells was placed in the presence of a defined concentration of allogenic T lymphocytes (mixed allogenic reaction) or autologous T lymphocytes (mixed autologous reaction) for 4 and 5 days, respectively. The proliferation of the T lymphocytes was determined by incorporation of tritiated thymidine. The experimental protocols for the mixed lymphocytic reactions are described in more detail in the article of K. Duperrier et al. cited above.

B. Results

Phenotype of the Dendritic Cells

The Tables 10 to 12 below summarize the percentages of expression of the different markers as well as the mean fluorescence intensity (MFI) of each marker (in parentheses).

TABLE No. 10

| Donor 1 | CD83 | CD80 | CD86 | CD40 | CD54 | HLA-DR | CD1a |
|---|---|---|---|---|---|---|---|
| TNF-α | 91% | 69.7% | 99.5% | 99.4% | 98.4% | 99.5% | 13.2% (28) |
| | (132) | (85) | (209) | (1084) | (195) | (803) | |
| LCOS 1013 | 84.6% | 93.2% | 99.7% | 99.7% | 99.2% | 99.7% | 10.8% (20) |
| | (157) | (134) | (214) | (1270) | (306) | (502) | |

TABLE No. 11

| Donor 2 | CD83 | CD80 | CD86 | CD40 | CD54 | HLA-DR | CD1a |
|---|---|---|---|---|---|---|---|
| TNF-α | 88% | 55.1% | 99.1% | 99.2% | 97.8% | 98.6% | 20.6% (215) |
| | (221) | (89) | (299) | (1104) | (355) | (1108) | |
| LCOS 1013 | 81.4% | 81.8% | 98.1% | 99.3% | 97.5% | 98.8% | 11.1% (299) |
| | (242) | (169) | (298) | (1441) | (416) | (1102) | |

TABLE No. 12

| | | | | | | HLA- | |
|---|---|---|---|---|---|---|---|
| Donor 3 | CD83 | CD80 | CD86 | CD40 | CD54 | DR | CD1a |
| TNF- | 91.8% | 48.7% | 98.5% | 98.7% | 95.9% | 98.9% | Neg. |
| α | (108) | (65) | (209) | (918) | (154) | (768) | |
| LCOS | 92.3% | 89.4% | 98.2% | 98.7% | 98.4% | 97.5% | Neg. |
| 1013 | (118) | (104) | (214) | (1270) | (288) | (588) | |

The dendritic cells generated in the presence of the molecule LCOS 1013 exhibit a phenotype characteristic of the mature dendritic cells, as a result of the expression of the CD83 molecules, of the co-stimulatory and adhesion molecules as well as the weak expression of CD1a.

It appears, however, that the percentage of expression, as well as the MFI of the molecule CD80 are increased in the presence of the molecule LCOS 1013, compared with TNF-α, as is the MFI of the molecules CD40 and CD54.

These results suggest a differential effect of the molecule LCOS 1013 compared with TNF-α as regards the maturation of the dendritic cells.

Functions of the Dendritic Cells

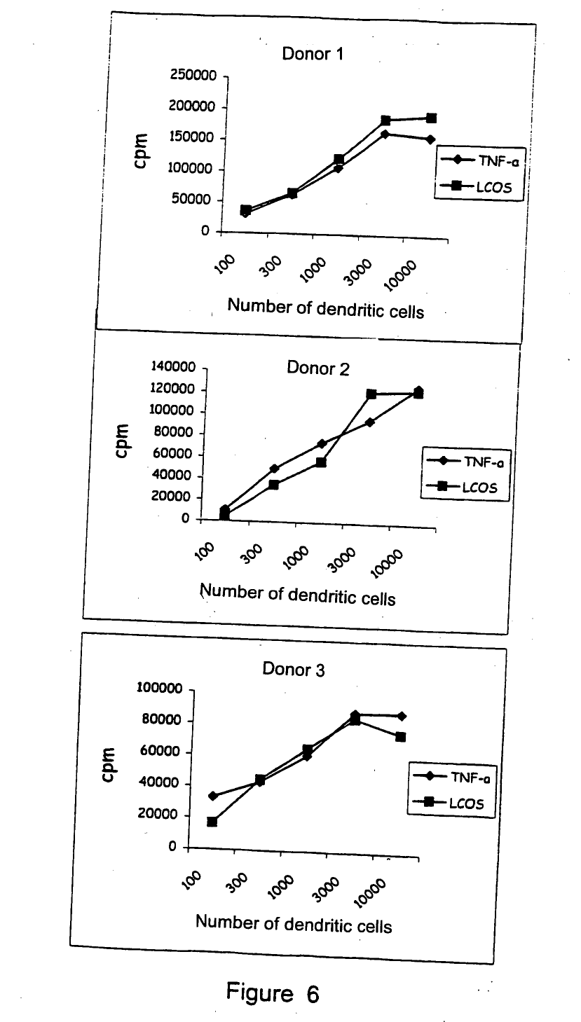

The results of the mixed allogenic lymphocytic reactions are illustrated in FIG. 6. They show that the dendritic cells cultivated in the presence of LCOS 1013 exhibit a high allostimulatory capacity, comparable to that of the cells generated in the presence of TNF-α.

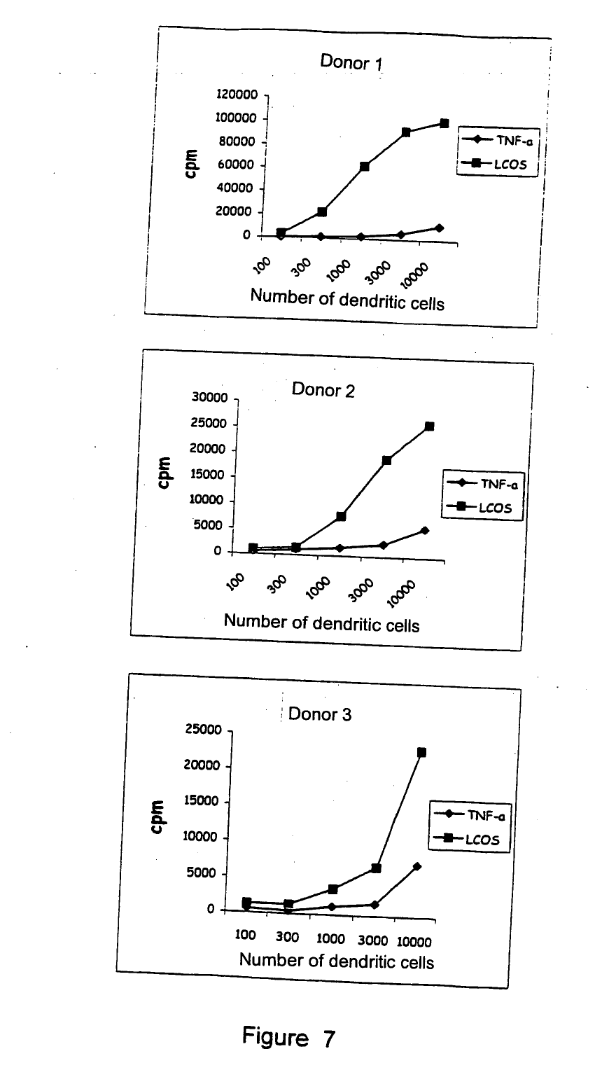

The results of the mixed autologous lymphocytic reactions are presented in FIG. 7. Remarkably, the dendritic cells cultivated in the presence of LCOS 1013 exhibit a capacity to stimulate autologous T lymphocytes much greater than the cells generated in the presence of TNF-α (at least 5 fold higher).

C. Conclusion

The results above show that the molecule LCOS 1013 induces in an efficacious manner the maturation of the dendritic cells generated from human monocytes, at the phenotypic and functional levels.

It is interesting to note however that the expression of the molecule CD80 is greatly increased in the presence of LCOS 1013 and that the cells induce a strong autologous response by comparison with TNF-α, and do this in the case of the 3 donors tested.

This is in agreement with the results presented by Scheinecker et al. (Journal of Immunology, 1998, 161: 3966-3973), which suggest that the molecule CD80 might play an essential role in the initiation of the mixed autologous reaction.

### EXAMPLE 12

Study of the Effect of Different Concentrations of LCOS 1013 on the Maturation of the Dendritic Cells.

The effect of different concentrations of LCOS 1013 on the maturation of the dendritic cells generated from human monocytes from a single donor was studied in comparison with TNF-α.

The concentrations tested for LCOS 1013 are 5, 10 and 25 μg/ml and 200 U/ml for TNF-α.

The results of the phenotypic studied are summarized in the following Table:

TABLE No. 13

| Donor 1 | HLA-DR | CD83 | CD54 | CD40 | CD86 | CD80 |
|---|---|---|---|---|---|---|
| TNF-α | 92.12% | 70.02% | 87.12% | 83.44% | 94.44% | 0.49% |
| (200 U/Ml) | 877.54 | 65.55 | 137.49 | 289.54 | 180.81 | 95.04 |
| LCOS 1013 | 81.69% | 76.35% | 89.39% | 75.02% | 93.33% | 3.26% |
| (5 μg/ml) | 545.30 | 119.62 | 229.59 | 454.18 | 197 | 90.79 |
| LCOS 1013 | 87.17% | 68.20% | 90.66% | 79.99% | 94.47% | 2.92% |
| (10 μg/ml) | 500.55 | 93.85 | 229.17 | 411.53 | 184.14 | 91.01 |
| LCOS 1013 | 94.52% | 66.35% | 93.15% | 91.22% | 91.07% | 1.07% |
| (25 μg/ml) | 504.84 | 105.29 | 237.68 | 394.41 | 191.62 | 99.64 |

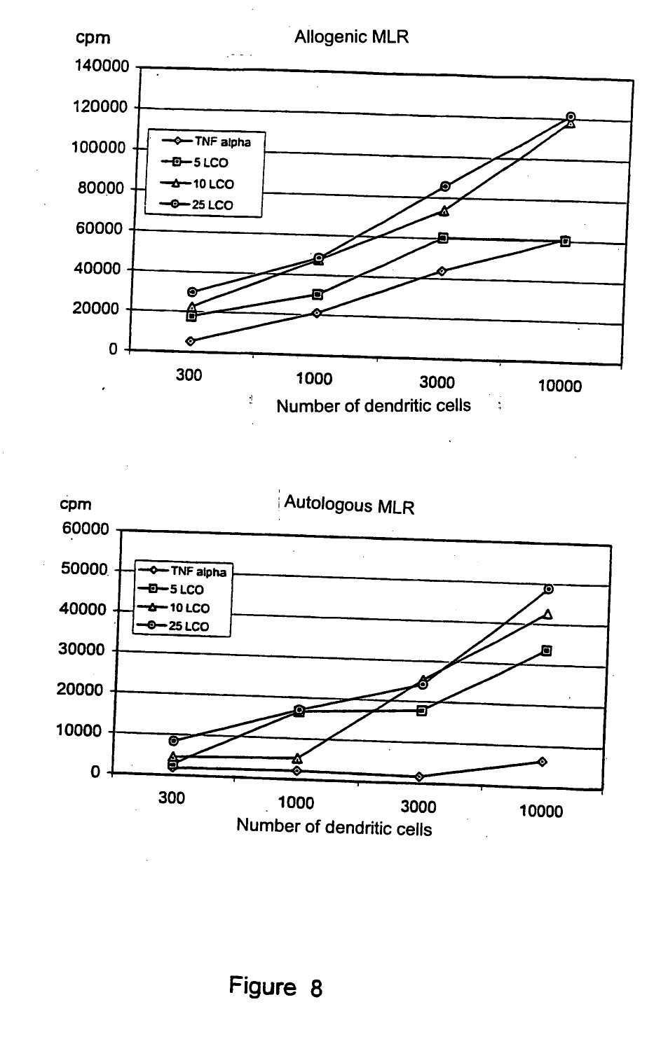

The functionality of the cells obtained was studied by means of mixed allogenic and autologous lymphocytic reactions, the results of which are presented in FIG. 8.

The dendritic cells cultivated in the presence of LCOS 1013, at the three concentrations tested, exhibit a high allostimulatory capacity, comparable to that of the cells generated in the presence of TNF-α.

There again and irrespective of the concentration of LCOS 1013 used, it is observed that the dendritic cells cultivated in the presence of LCOS 1013 exhibit a capacity to stimulate autologous T lymphocytes much greater than the cells generated in the presence of TNF-α.

1. Process for obtaining mature dendritic cells or activated macrophages from monocytes, monocyte precursors or hematopoietic stem cells, characterized in that the said monocytes, precursors or stem cells are placed in contact with RU 41740 or an analogue of the latter, this compound being selected such that the placing in contact of immature dendritic cells with the said compound makes possible the functional maturation of the dendritic cells, demonstrated by their capacity
to trigger a primary response in vitro against an infectious or tumor antigen placed in contact with the dendritic cells beforehand and/or during their culture with the T lymphocytes;
to induce the proliferation of T lymphocytes in mixed autologous or allogenic culture.

2. Process for obtaining mature dendritic cells or activated macrophages from monocytes, monocyte precursors or hematopoietic stem cells, characterized in that the said monocytes, precursors or stem cells are placed in contact with RU 41740 or an analogue of the latter, this compound being selected such that the placing in contact of immature dendritic cells with the said compound makes possible the phenotypic maturation of the dendritic cells, demonstrated by a significant increase in the expression of the molecules CD40, CD83, CD86 and HLA-DR and a very marked diminution in the expression of the molecules CD14 and CD1a by the said dendritic cells.

3. Process according to claim 1 or 2, characterized in that the monocytes, precursors or stem cells are placed in contact with an analogue of RU 41740 obtained from the strain O₁K₂NCTC 5055 of Klebsiella pneumoniae.

4. Process for obtaining mature dendritic cells presenting selected antigens, starting from monocytes, monocyte precursors or hematopoietic stem cells, characterized in that the said precursors are placed in contact with RU 41740 or an analogue thereof, coupled to molecules comprising the said antigens.

5. Process according to claim 4, characterized in that the coupling between RU 41740 or its analogue and the antigens is non-covalent.

6. Process according to one of the claims 1, 2, 4 and 5, in which the compound placed in contact with the monocytes, monocyte precursors or hematopoietic stem cells is RU 41740, coupled or not to antigenic molecules.

7. Process according to claim 6, in which RU 41740 is added to the culture medium of the monocytes, monocyte precursors or hematopoietic stem cells at a final concentration comprised between 1 ng/ml and 1 mg/ml, preferentially between 100 ng/ml and 10 μg/ml.

8. Process according to claim 3, characterized in that the analogue of RU 41740 is LCOS 1013 or LCOS 1014.

9. Process according to claim 8, in which LCOS 1013 or LCOS 1014 is added to the culture medium of the monocytes, monocyte precursors or hematopoietic stem cells at a final concentration comprised between 1 ng/ml and 1 mg/ml, preferentially between 100 ng/ml and 50 μg/ml.

10. Process according to one of the claims 1 to 9, in which the dendritic cells are treated ex vivo for the preparation of a medicine destined for the prophylaxis, attenuation or treatment of cancerous, infectious, allergic or auto-immune diseases.

11. Use of RU 41740 or an analogue thereof for the preparation of a composition containing mature dendritic cells and/or activated macrophages.

12. Use of RU 41740 or an analogue thereof for the preparation of a pharmaceutical composition for topical administration, destined to promote the maturation of the Langerhans cells of the skin.

13. Use of a coupling product between RU 41740 or an analogue thereof and one or more antigens, for the preparation of a composition able to induce the production of mature dendritic cells or activated macrophages presenting the said antigens.

14. Use of dendritic cells obtained by means of a process according to one of the claims 1 to 10 in the manufacture of a composition able to promote an anti-tumor immune response.

15. Use of dendritic cells obtained by means of a process according to one of the claims 1 to 10, in the manufacture of a composition able to promote an immune response against an infection by a micro-organism.

16. Use of dendritic cells obtained by means of a process according to one of the claims 1 to 10 and incubated in the presence of an immunosuppressant, in the manufacture of a composition able to modify the immune response in the sense of a tolerance.

17. Use of dendritic cells obtained by means of a process according to one of the claims 1 to 10, for the detection and/or characterization of the histocompatibility antigens.

18. Coupling product between RU 41740 or an analogue thereof and antigenic molecules for inducing the maturation of dendritic cells or the activation of macrophages.

19. Coupling product according to claim 18, characterized in that RU 41740 or its analogue is linked to antigenic molecules by means of non-covalent bonds.

20. Coupling product according to claim 18 or 19, characterized in that the antigenic molecules are non-protein in nature.
